(19) United States
(12) Patent Application Publication
(10) Pub. No.: US 2010/0221833 A1
(43) Pub. Date: Sep. 2, 2010

(54) PROCESS FOR DIFFERENTIATION OF VASCULAR ENDOTHELIAL PROGENITOR CELLS FROM EMBRYOID BODIES DERIVED FROM EMBRYONIC STEM CELLS USING HYPOXIC MEDIA CONDITION

(75) Inventors: Hyung-Min Chung, Gyeonggi-do (KR); Ju-Mi Kim, Gyeonggi-do (KR); Soo-Hong Lee, Seoul (KR); Sung-Hwan Moon, Gyeonggi-do (KR)

Correspondence Address: THE NATH LAW GROUP, 112 South West Street, Alexandria, VA, 22314, US

(73) Assignees: CHABIO & DIOSTECH CO., LTD., Yongin-city, Kyunggi-do (KR); COLLEGE OF MEDICINE POCHON CHA UNIVERSITY INDUSTRY-ACADEMIC COOPERATION FOUNDATION,, Pochon-siu, Gyeonggi-do (KR)

(21) Appl. No.: 12/733,661

(22) PCT Filed: Aug. 14, 2008
(86) PCT No.: PCT/KR2008/004723

§ 371 Date: Mar. 12, 2010

(30) Foreign Application Priority Data
Sep. 14, 2007 (KR) 10-2007-0093418

(51) Int. Cl. C12N 5/0735

(52) U.S. Cl. 435/377

(57) ABSTRACT

The present invention provides a process for differentiation of vascular endothelial progenitor cells from embryoid bodies derived from embryonic stem cells, the process comprising: (a) treating a culture medium comprising embryoid bodies derived from embryonic stem cells such that the concentration of oxygen dissolved in the culture medium is in the range of about 1 ppm to about 5 ppm; (b) culturing the culture medium prepared in step (a) in an incubator in which the oxygen (O₂) tension is equal to or less than about 15% to differentiate the embryoid bodies into vascular endothelial progenitor cells; and (c) isolating the vascular endothelial progenitor cells from the culture medium obtained in step (b).

### TECHNICAL FIELD

The present invention relates to a process for differentiation of vascular endothelial progenitor cells from embryoid bodies derived from embryonic stem cells, and more particularly, to a process for differentiation and isolation of vascular endothelial progenitor cells in high yield and high purity, using hypoxic media condition by directly inducing hypoxia condition in a culture medium including embryoid bodies derived from embryonic stem cells.

### BACKGROUND ART

Human embryonic stem cells retain infinite self-renewality and pluripotency which can be differentiated into three germ cell layers (endodermal, ectodermal, and mesodermal) which organize a human body (Thomson J A, Itskovitz-Eldor J, Shapiro S S, Waknitz M A, Swiergiel J J, Marshall V S, Jones J M, Embryonic stem cell lines derived from human blastocysts. Science (1998) 282:1145-1147). With recent successful studies on differentiation of human embryonic stem cells into neural cells, endothelial cells, muscle cells, pancreatic cells, or the like, the human embryonic stem cells are considered to play a critical role in studies of therapy for incurable diseases.

Thus, development of high-purity cell therapy, through methods for differentiating human embryonic stem cells into specific cells and methods for isolation of the differentiated cells, will offer critical techniques for incurable diseases and breakthrough and primitive therapy, as a novel therapy having excellent advantages compared with conventional treatments based on drugs and surgery.

Cell therapy using vascular endothelial progenitor cells is considered to be directly or indirectly applied to rapidly increasing vascular diseases such as cardiovascular diseases, cerebrovascular diseases, and diabetic ulcer.

However, vascular endothelial progenitor cells obtained using conventional differentiation and isolation techniques coexist with undesirable cells and undifferentiated cells. Thus, there is a need to develop a process for differentiation of vascular endothelial progenitor cells and isolation of the differentiated vascular endothelial progenitor cells in high yield and high purity.

According to studies conducted by Levenberg S, et al., embryoid bodies to cultivated from human embryonic stem cells spontaneously differentiate into vascular endothelial cells, and the vascular endothelial cells are isolated from the differentiated embryoid bodies using a fluorescence activated cell sorter (FACS) (Levenberg S, Golub J S, Amit M, Itskovitz-Eldor J, Langer R. PNAS (2002) 99, 4391-4396). However, the efficiency of differentiation was too low, showing that only 2% of embryoid bodies are differentiated into the vascular endothelial cells.

Induction of hypoxia condition has been reported as one of the efficient ways for differentiation. Diana L. Ramiez-Bergeron et al. reported that hypoxia condition promotes differentiation of embryoid bodies into mesodermal cells and hemangioblasts (Diana L. Ramiez-Bergeron, Anja Runge, Karen D. Cowden Dahl, Hans Joerg Fehling, Gordon Keller and M. Celeste Simon1, Hypoxia affects mesoderm and enhances hemangioblast specification during early development. Development (2004) 131, 4623-4634).

A method of inducing hypoxia condition can be classified into a method of using a hypoxia incubator, a method of using a commercially available gas pack (e.g., Becton Dickinson/BBL gas generator), and a method of using a reagent such as CoCl₂ and defferrioxamine (DFX). However, in case of using the hypoxia incubator, hypoxia condition is not directly induced in a culture medium since the hypoxia condition is only physically induced. And also, it is difficult to continuously maintain the hypoxia condition at the initial stage and to directly measure the degree of hypoxic induction. In case of using gas pack, hypoxia condition is induced only physically, which is similar to using a hypoxia incubator, and also the hypoxia condition is induced only one time. In addition, since the gas pack can induce only hypoxic condition having oxygen (O₂) tension of 1% or less, it can be used only in cells that can grow in the hypoxic condition having oxygen (O₂) tension of 1% or less. Even though chemical reagents such as CoCl₂ and DFX can induce hypoxia condition at a molecular level by influencing cell signaling mechanism, the chemical reagents may be toxic to the cells, and thus the use thereof is limited. As described above, according to the conventional methods of inducing hypoxic culture media for differentiation and culture of cells, there are various problems such as unstable hypoxia condition maintenance, cell toxicity and restrictive use in cells. Furthermore, the proposed methods in the prior arts have defects such as low efficiency of differentiation of the vascular endothelial progenitor cells and low purity thereof.

In particular, Wang G L, et al. have reported that initial 4-6 hours after exposed in hypoxia condition are important for expression of hypoxia-inducible factor (HIF)-1α which is a mediator of hypoxic response in human bodies (Wang G L. Semenza G L. General involvement of hypoxia-inducible factor 1 in transcriptional response to hypoxia. PNAS .May 1; 90(9):4304-8. 1993). Accordingly, vascular endothelial progenitor cells may not be obtained with high yield by merely incubating the culture medium in a hypoxia incubator.

Therefore, there is a need to develop a process for differentiation of vascular endothelial progenitor cells with high yield and high purity in order to develop cell therapy for incurable vascular diseases.

### DETAILED DESCRIPTION OF THE INVENTION

### Technical Problem

The present inventors conducted extensive research to develop a process for differentiation of vascular endothelial progenitor cells from embryoid bodies derived from embryonic stem cells and isolation of the differentiated vascular endothelial progenitor cells in high yield and high purity. As a result, the present inventors have found that efficiency of differentiation into vascular endothelial progenitor cells is significantly increased when the environment surrounding embryoid bodies is directly exposed to hypoxia condition by directly inducing hypoxia condition in a culture medium including embryoid bodies derived from embryonic stem cells. Furthermore, pure vascular endothelial progenitor cells can be isolated by isolating cells positive for both CD133 and KDR/Flk-1 (i.e., double positive population) using a FACS.

Thus, the present invention provides a process for differentiation of vascular endothelial progenitor cells from embryoid bodies derived from embryonic stem cells and isolation of the differentiated vascular endothelial progenitor cells in high yield and high purity.

### Technical Solution

According to an aspect of the present invention, there is provided a process for differentiation of vascular endothelial progenitor cells from embryoid bodies derived from embryonic stem cells, the process comprising: (a) treating a culture medium comprising embryoid bodies derived from embryonic stem cells such that the concentration of oxygen dissolved in the culture medium is in the range of about 1 ppm to about 5 ppm; (b) culturing the culture medium prepared in step (a) in an incubator in which the oxygen (O₂) tension is equal to or less than about 15% to differentiate the embryoid bodies into vascular endothelial progenitor cells; and (c) isolating the vascular endothelial progenitor cells from the culture medium obtained in step (b).

In step (a), the concentration of oxygen dissolved in the culture medium may be about 2 ppm to about 3 ppm, and the treatment may be performed by bubbling an inert gas, such as argon (Ar), helium (He), or nitrogen (N₂) gas, more preferably nitrogen (N₂) gas.

In step (b), the oxygen tension may be about 1% to about 10% and the culturing may be performed for about 1 week to 3 weeks.

In step (c), the isolation may be performed using a fluorescence activated cell sorter (FACS). The isolation using the FACS may be performed by isolating cells positive for both CD133 and KDR/Flk-1 as markers

### ADVANTAGEOUS EFFECTS

The process of the present invention includes treating a culture medium comprising embryoid bodies derived from embryonic stem cells so as to result in hypoxia condition (e.g., such that the concentration of dissolved oxygen is in the range of 1 to 5 ppm), thereby the environment surrounding embryoid bodies being directly exposed to hypoxia condition; thus efficiency of differentiation into vascular endothelial progenitor cells can be significantly increased.

And, according to the present invention, hypoxia condition can be simply, to directly, stably, and efficiently induced using bubbling of an inert gas such as nitrogen (N₂) gas. In particular, since the bubbling of the inert gas such as nitrogen (N₂) gas does not cause denaturation of proteins existing in a culture medium and is not toxic to cells, problems that may be caused by a chemical reagent such as CoCl₂ and DFX do not occur.

When, according to the present invention, cells positive for both CD133 and KDR/Flk-1 (i.e., double positive population) are isolated by a FACS using a double marker (that is, CD133 and KDR/Flk-1 double markers), instead of a single marker, vascular endothelial progenitor cells can be isolated with high purity. Therefore, the vascular endothelial progenitor cells can be isolated with high yield and high purity according to the process of the present invention.

### BRIEF DESCRIPTION OF THE DRAWINGS

FIG. 1 shows photographs of a nitrogen (N₂) gas regulator, a dissolved oxygen (DO) meter measuring dissolved oxygen levels in a culture medium, and a hypoxia incubator.

FIG. 2 schematically illustrates nitrogen (N₂) gas bubbling.

FIG. 3 is a graph illustrating the results of measurement of the DO amounts in various conditions.

FIG. 4 is a graph illustrating assay results of expression of vascular endothelial progenitor cell markers in embryoid bodies differentiated in culture media cultured in normoxia conditions and hypoxia conditions, using a FACS.

FIG. 5 illustrates images of an immunostained vascular endothelial progenitor cell marker PECAM expressed in embryoid bodies cultured in culture media in normoxia conditions and hypoxia conditions.

FIG. 6 illustrates relative amount of vascular endothelial progenitor cells, which are positive for both CD133 and KDR, in embryoid bodies cultured in culture media in normoxia conditions and hypoxia conditions, measured using a FACS.

FIG. 7 show optical microscope images of vascular endothelial progenitor cells isolated from embryoid bodies cultured in hypoxia conditions using a FACS and cord blood-derived vascular endothelial progenitor cells.

FIG. 8 illustrates optical microscope images of vascular endothelial progenitor cells isolated using a FACS and results of RT-PCR, matrigel assay, and ac-LDL uptake assay, in order to verify the characteristics as vascular endothelial cells.

FIG. 9 illustrates cell cycles of each of isolated vascular endothelial progenitor cells and undifferentiated human embryonic stem cells, and the amount of expression of the undifferentiation marker SSEA-4, measured using a FACS.

FIG. 10 illustrates DNA fingerprintings of isolated vascular endothelial progenitor cells and undifferentiated human embryonic stem cells.

FIG. 11 illustrates photographs showing morphological features of vascular endothelial progenitor cells isolated from a differentiated culture medium in hypoxia conditions using specific markers CD133, KDR, and CD133/KDR, and cord blood-derived vascular endothelial progenitor cells.

### BEST MODE FOR CARRYING OUT THE INVENTION

The “embryoid body” used herein is a cell mass spontaneously differentiated from embryonic stem cells and refers to a cell aggregate which can differentiate into three germ layers (endodermal, ectodermal, and mesodermal). The embryoid body can be maintained in an appropriate culture medium.

In addition, the “vascular endothelial progenitor cell” is a progenitor cell which can differentiate into vascular endothelial cell forming endothelium of blood vessel, that is, a cell having progenitor features. The vascular endothelial progenitor cell express a cell surface marker of the vascular endothelial cell marker such as PECAM, CD34, KDR/Flk-1, CD133, Tie-2, VE-cadherin, and vWF.

The process of differentiation according to the present invention includes treating a culture medium comprising embryoid bodies derived from embryonic stem cells such that the concentration of oxygen dissolved in the culture medium is in the range of about 1 ppm to about 5 ppm [step (a)].

The embryonic stem cells used herein may be any embryonic stem cells derived from a mammal, and preferably embryonic stem cells derived from human. The human embryonic stem cells (hESCs) are pluripotent cells derived from the inner cell mass of human blastocysts. For example, the hESCs may be CHA-hES3 (Ahn S E, Kim S, Park K H, Moon S H, Lee H J, Kim G J, Lee Y J, Park K H1 Cha K Y, Chung H M. “Primary bone-derived cells induce osteogenic differentiation without exogenous factors in human embryonic stem cells”, Biochem Biophys Res Commun. (2006) 10; 340(2): 403-408), but is not limited thereto. In addition, the hESCs may be easily established by those skilled in the art.

The embryoid body may be derived from hESCs using a known method. For example, the embryoid body may be derived from hESCs by culturing the hESCs in a DMEM/F12 (Dulbecco's modified Eagle's medium; GIBCO) supplemented with serum (or serum replacement), L-glutamine, nonessential amino acid, and β-mercaptoethanol according to a method introduced by Kim J, Moon S H, Lee S H, Lee D Y, Koh G Y and Chung H M, “Effective Isolation and Culture of Endothelial Cells in Embryoid Body Differentiated from Human Embryonic Stem Cells” Stem cell and Development (2007) 16:269.280.

In the process for differentiation according to the present invention, the culture medium including embryoid bodies derived from the embryonic stem cells may be any culture medium in which embryoid bodies can spontaneously differentiate into the vascular endothelial progenitor cells, for example, a culture medium used in Kim J, Moon S H, Lee S H, Lee D Y, Koh G Y and Chung H M, “Effective Isolation and Culture of Endothelial Cells in Embryoid Body Differentiated from Human Embryonic Stem Cells” Stem cell and Development (2007) 16:269.280. For example, the DMEM/F12 (Dulbecco's modified Eagle's medium; GIBCO) supplemented with serum (or serum replacement), L-glutamine, nonessential amino acid, and β-mercaptoethanol may be used, but the culture medium is not limited thereto.

The concentration of oxygen dissolved in the culture medium including embryoid bodies derived from the embryonic stem cells may be in the range of about 1 ppm to about 5 ppm, preferably about 2 ppm to about 3 ppm, and more preferably about 2 ppm.

The treatment in step (a) may be performed by bubbling an inert gas, such as argon (Ar), helium (He), and nitrogen (N₂) gas, and preferably nitrogen (N₂) gas, but is not limited thereto if the concentration of oxygen dissolved in the culture medium is within the range described above. The bubbling of the inert gas such as nitrogen (N₂) gas may directly and efficiently induce hypoxia conditions in the culture medium by removing oxygen contained in the culture medium. In addition, the inert gas such as is nitrogen (N₂) gas does not easily react with other compounds at room temperature due to its stable chemical structure, and thus the hypoxia conditions may be stably maintained in the culture medium without denaturation of nutrients contained in the culture medium.

For example, hypoxia conditions may be induced in the culture medium including embryoid bodies derived from embryonic stem cells by simply bubbling nitrogen (N₂) gas in the culture medium. The induction of hypoxia conditions may be identified by measuring the concentration of dissolved oxygen using a DO-meter. In general, hypoxia conditions may be induced by bubbling nitrogen (N₂) gas for 5 minutes in 50 mL of culture medium.

The process for differentiation according to the present invention includes differentiating the embryoid bodies into vascular endothelial progenitor cells by culturing the culture medium, in which hypoxia conditions are induced as described above, in an incubator in which the oxygen (O₂) tension is equal to or less than about 15%, preferably in the range of 1 to 10%, and more preferably about 3% [step (b)].

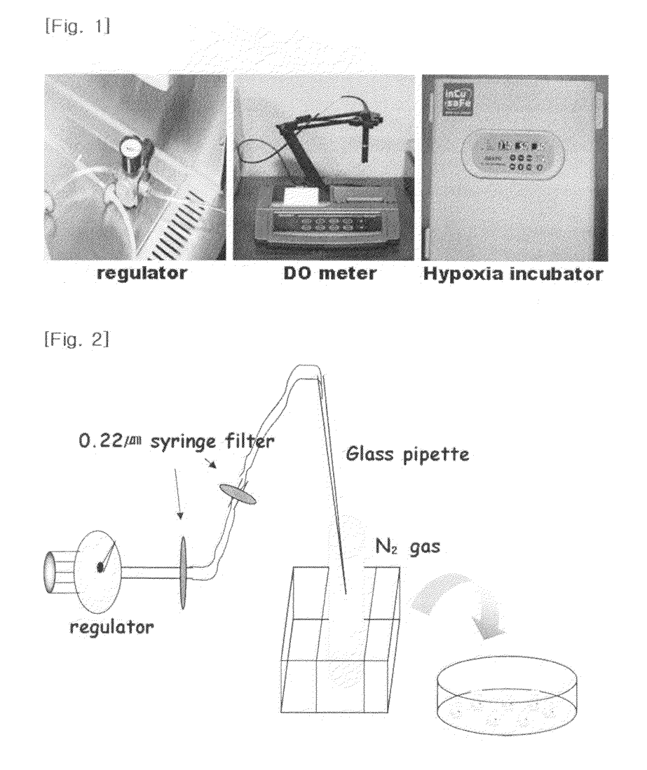

The cultivation may be performed using a hypoxia incubator as shown in FIG. 1. However, any incubator in which hypoxia conditions may be maintained, that is, if the oxygen partial pressure is equal to or less than 15%, preferably in the range of 1 to 10%, and more preferably about 3%, can be used, and the shapes and types of the incubator are not limited.

The cultivation may be performed for 1 to 3 weeks, and preferably for about 2 weeks, but the duration is not limited thereto. The culture medium may be changed, e.g., every 24 hours.

The process for differentiation according to the present invention includes (c) isolating the vascular endothelial progenitor cells from the culture medium, specifically from embryoid bodies in the culture medium, cultured in hypoxia conditions described above [step (c)].

The isolation may be performed using a conventional method, such as a physical isolation method, an enzymatic isolation method, a physical or enzymatic isolation after co-culture with other feeder cells. However, the isolation may be performed using a fluorescence activated cell sorter (FACS) in consideration of the purity of obtained vascular endothelial progenitor cells. In particular, if cells positive for a double marker instead of a single marker, that is, positive for both CD133 and KDR/Flk-1 (i.e., double positive population) are isolated using the FACS, purity of the vascular endothelial progenitor cells may be increased.

For example, the isolation of the double positive population using the FACS and using the CD133 and KDR/Flk-1 double marker may be performed as follows. Embryoid bodies are processed with an enzyme such as trypsin-EDTA to prepare a single cell, and stained using CD133 and KDR/Flk-1 in a buffer solution such as phosphate buffered saline supplemented with about 5% fetal bovine serum (FBS). Then, cells positive for both CD133 and KDR/Flk-1 are isolated using a FACS.

The vascular endothelial progenitor cells prepared as described above may be proliferated by subculturing them using a conventional culture medium for vascular endothelial cells, for example, an EGM-2/MV culture medium (Cambrex), trypsin-EDTA, or the like.

Hereinafter, the present invention will be described more specifically with reference to the following examples. The following examples are only for illustrative purposes and are not intended to limit the scope of the invention.

### Example 1

### (1) Formation of Embryoid Body

Mouse fibroblast cells (STO cells, 7.0×10⁵ cells/60 mm dish) treated with mitomycin-c for 2 hours so as to inhibit proliferation were cultured for 24 hours in a gelatin-coated culture dish including a culture medium for feeder cells (90% DMEM high glucose medium supplemented with 10% fetal bovine serum (FBS), 0.1 mM mercaptoethanol, and 1% non-essential amino acid (Gibco)). The culture dish was washed once with DMEM/F-12, and the culture medium was replaced by 80% to DMEM/F-12 supplemented with 20% serum replacement (SR), 0.1 mM mercaptoethanol, 1% non-essential amino acid (Gibco), and 4 ng/ml of basic fibroblast growth factor (bFGF). Human embryonic stem cells (CHA-hES3) were cultured in the culture medium for about 7 days. Colonies of the hESCs formed in the culture medium were isolated from feeder cells using a glass pipette having a predetermined shape by heating with an alcohol lamp, and suspension-cultured in an embryonic stem cell culture medium, in which the bFGF is removed, for about 2 days to form embryoid bodies.

### (2) Induction of Hypoxia Conditions

50 mL of the culture medium including the embryoid bodies (i.e., the embryonic stem cell culture medium in which the bFGF is removed) was bubbled using nitrogen (N₂) gas for about 5 minutes to induce hypoxia conditions. A regulator was connected to the culture medium in order to regulate the amount of gas inflow, and a 0.22 μm syringe filters were respectively installed in the regulator and at the central region of the glass pipette which contacts the culture medium in order to prevent contamination of the culture medium, and then, bubbling was performed. Then, the amount of dissolved oxygen in the culture medium in which nitrogen (N₂) gas bubbling was completed was measured using a DO meter. The culture medium including the embryoid bodies in which the hypoxia conditions are induced was cultured for 21 days in a hypoxia incubator in which oxygen (O₂) tension was about 3%. FIGS. 1 and 2 illustrate nitrogen (N₂) gas bubbling and devices used therefor.

### (3) Isolation of Vascular Endothelial Progenitor Cells

The expression amounts of vascular endothelial progenitor cell markers such as CD133, CD34, and KDR/Flk-1 were measured every 7 days during the culture period (21 days) using a FACS. The expression of a vascular endothelial progenitor cell marker PECAM in the embryoid bodies was identified in normoxia conditions and hypoxia conditions using immunostaining. The expression amount of CD133 and KDR/Flk-1 double positive populations was measured using a FACS and then the CD133 and KDR/Flk-1 double positive populations were isolated using the FACS.

### (4) Subculture

The isolated cells were cultured in EGM-2/MV culture medium (Cambrex). When the concentration of the cells distributed on a culture dish was about 70 to 80%, the cells were isolated from the culture dish using trypsin-EDTA (Gibco) and then subcultured.

### Experimental Example 1

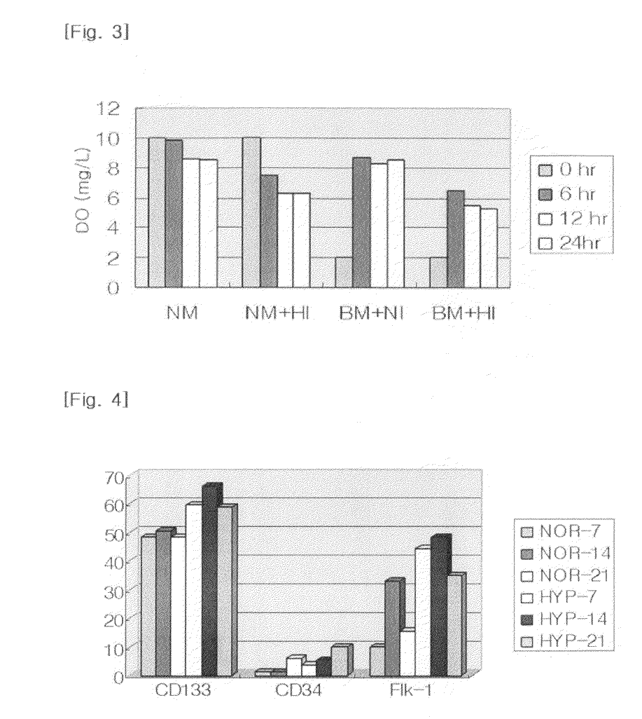

The amounts of dissolved oxygen were measured in various conditions described below in the culture medium described in (2) of Example 1. That is, the culture medium including embryoid bodies prepared in (1) of Example 1 was cultured in conditions regulated by combining a normoxia medium, a hypoxia medium, a normoxia incubator, and a hypoxia incubator as shown in Table 1, and then the amounts of dissolved oxygen were measured with time. The results are shown in Table 1 and FIG. 3. The dissolved oxygen was measured in ppm.

TABLE 1

| | NM + NI | NM + HI | BM + NI | BM + HI |
|---|---|---|---|---|
| 0 hour | 10 | 10 | 2 | 2 |
| 6 hours | 9.8 | 7.5 | 8.7 | 6.5 |
| 12 hours | 8.6 | 6.3 | 8.3 | 5.5 |
| 24 hours | 8.5 | 6.3 | 8.5 | 5.3 |

NM: normoxia medium
BM: hypoxia medium
NI: normoxia incubator
HI: hypoxia incubator

As shown in Table 1 and FIG. 3, when a hypoxic culture medium bubbled with nitrogen (N₂) gas was cultured in a hypoxia incubator, oxygen (O₂) tension was the most significantly decreased to a hypoxic condition within initial 4-6 hours. Since initial 4-6 hours of hypoxic exposure is important for the expression of hypoxia-inducible factor (HIF)-1 α which is a mediator of hypoxic response in human bodies (Wang G L. Semenza G L. General involvement of hypoxia-inducible factor 1 in transcriptional response to hypoxia. PNAS. May 1; 90(9): 4304-8, 1993), the above-described results show that vascular endothelial progenitor cells can be obtained with high yield according to the process for differentiation according to the present invention.

### Experimental Example 2

The culture medium was cultured in the same manner as in Example 1, except is that nitrogen (N₂) gas bubbling was not performed in (2) of Example 1, and vascular endothelial progenitor cells were isolated (Comparative Example 1). The amounts of expression of vascular endothelial progenitor cell markers such as CD133, CD34, and KDR/Flk-1 in Example 1 and Comparative Example 1 were respectively measured every 7 days during the culture period (21 days) using a FACS. The results are shown in FIG. 4. As shown in FIG. 4, the amount of vascular endothelial progenitor cell marker expressed in embryoid bodies cultured in hypoxia conditions according to the present invention was greater than that of vascular endothelial progenitor cell marker expressed in embryoid bodies cultured in normoxia conditions. Furthermore, the expression amount of CD133 and KDR/Flk-1 was relatively greater in embryoid bodies differentiated for 14 days.

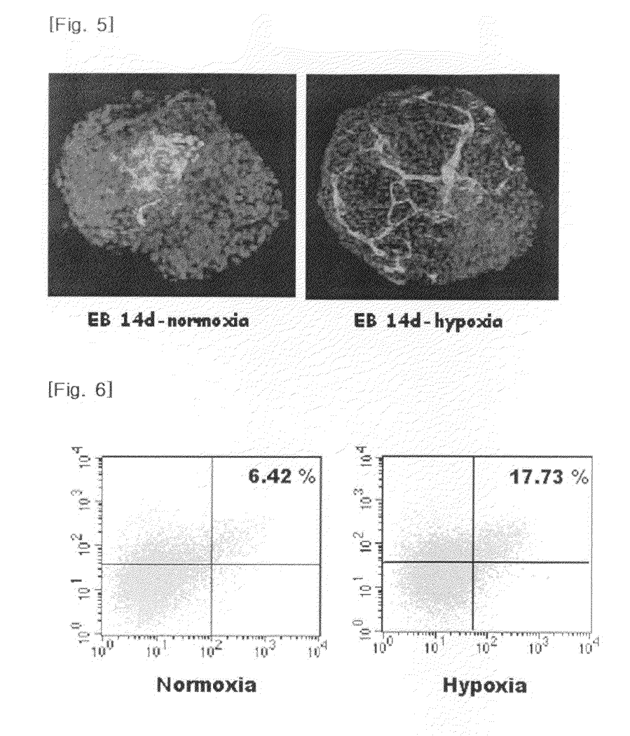

The expression of PECAM, one of the vascular endothelial progenitor cell markers, was identified in the embryoid bodies cultured for 14 days using immunostaining. As a result, the amount of the marker expressed in embryoid bodies cultured in hypoxia conditions was greater than that cultured in normoxia conditions, and distribution of the expression of the markers cultured in hypoxia conditions was larger than that cultured in normoxia conditions as shown in FIG. 5. In addition, as a result of analysis of embryoid bodies prepared in Example 1 and Comparative Example 1 using a FACS, the amount of CD133 and KDR/Flk-1 double positive population expressed in embryoid bodies differentiated in hypoxia culture medium was significantly increased, as shown in FIG. 6.

### Experimental Example 3

CD133 single positive population and KDR/Flk-1 single positive population were isolated from culture medium of (3) of Example 1 using a FACS (Comparative Example 2).

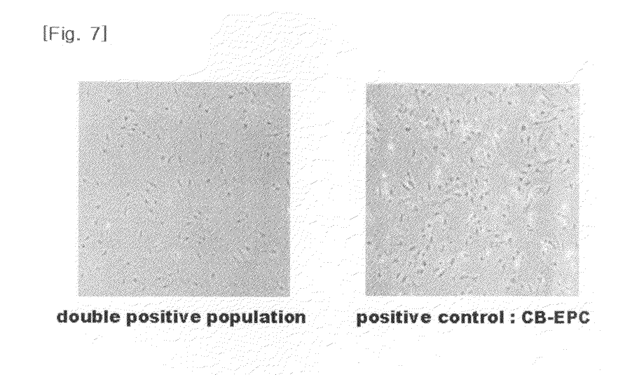

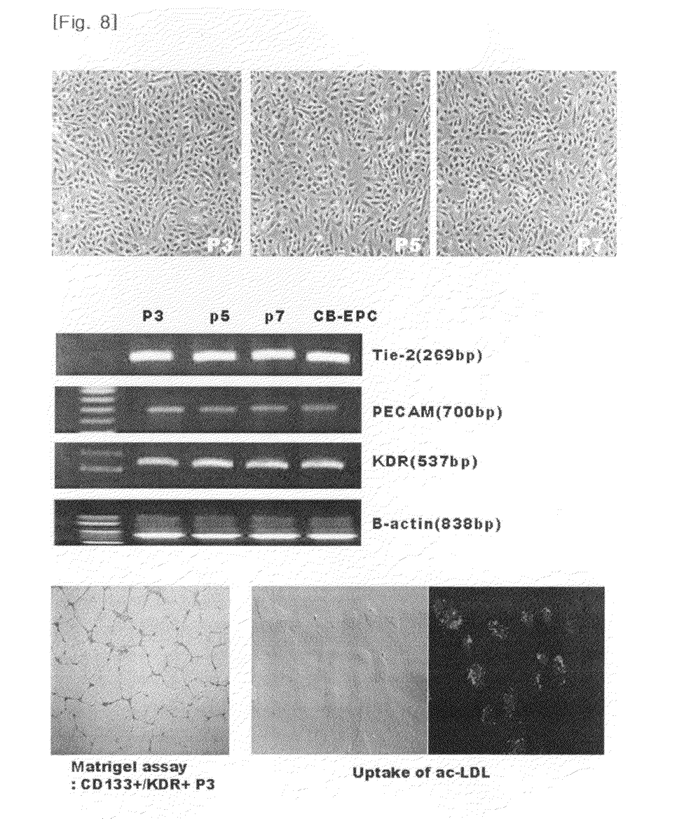

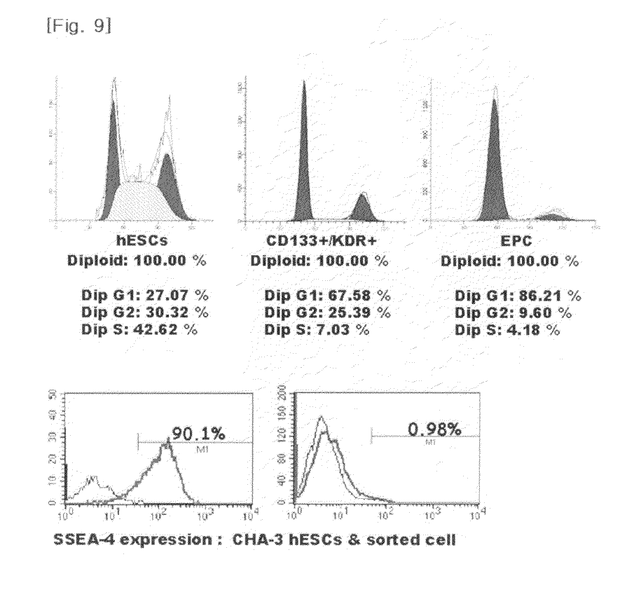

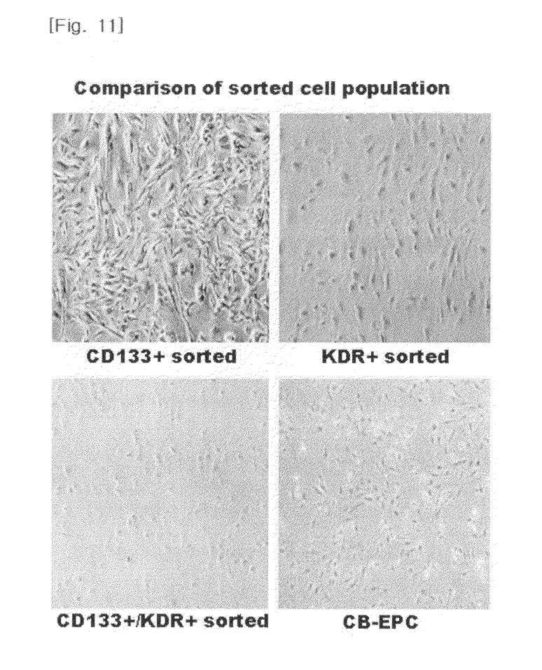

FIG. 7 show optical microscope images of cells isolated in Example 1 and Comparative Example 2 As shown in FIG. 7, CD133 and KDR/Flk-1 double positive population has similar morphological features to those of cord blood derived endothelial progenitor cells (positive control of FIG. 7). In addition, the isolation of CD133/KDR double positive population is more morphologically similar to that of cord blood-derived vascular endothelial progenitor cells compared to the isolation of CD133 or KDR/Flk-1 single positive population as described in Comparative Example 2 (FIG. 11).

### Experimental Example 4

Reverse transcriptase-polymerase chain reaction (RT-PCR), matrigel assay, and acetylated-low density lipoprotein (ac-LDL) uptake assay of CD133 and KDR/Flk-1 double positive population prepared in Example 1 were performed to identify features as the vascular endothelial progenitor cells.

As shown in FIG. 8, CD133 and KDR/Flk-1 double positive population is cultured and proliferated similarly to cord blood-derived vascular endothelial progenitor cells. It was identified that the expression of vascular endothelial progenitor cell markers, Tie-2, PECAM, and KDR was continued in subculture by RT-PCR introduced by Kim J, et al. Effective Isolation and Culture of Endothelial Cells in Embryoid Body Differentiated from Human Embryonic Stem Cells Stem cell and Development (2007) 16:269.280. In addition, the expression of the vascular endothelial progenitor cell markers, Tie-2, PECAM, and KDR was similar to the control group of the cord blood-derived vascular endothelial progenitor cells. Furthermore, it was identified that the isolated vascular endothelial cells formed a structure similar to blood vessels in three-dimensional culture environments of matrigel assay to evaluate the function of vascular endothelial cells derived from human embryonic stem cells, and that ac-LDL was taken up by the cells.

### Experimental Example 5

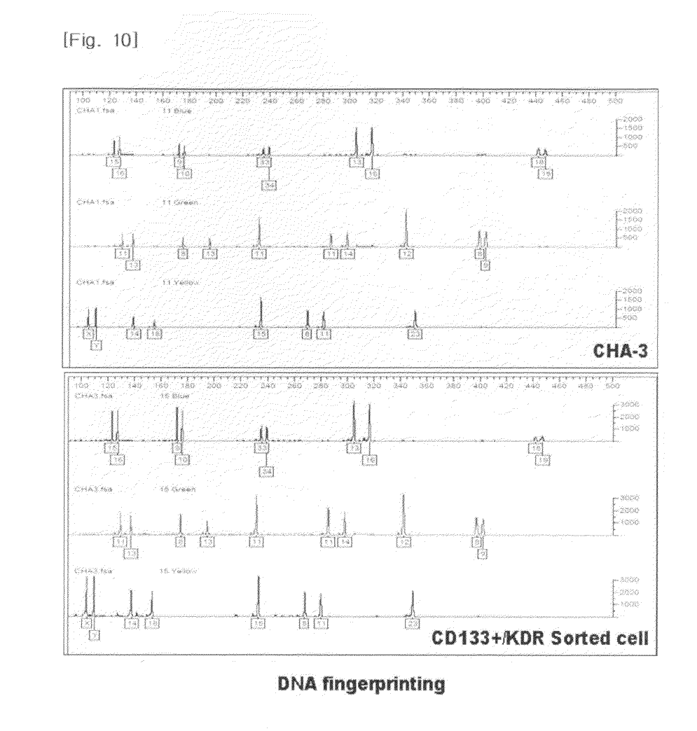

Cell cycles and expressions of undifferentiated markers were measured using a FACS in order to identify that the CD133 and KDR/Flk-1 double positive population prepared in Example 1 is differentiated. When the vascular endothelial progenitor cells were isolated, DNA fingerprinting was performed in order to identify that the vascular endothelial progenitor cells were derived from human embryonic stem cells. The results are shown in FIGS. 9 and 10.

As shown in FIGS. 9 and 10, the majority of isolated vascular endothelial progenitor cells stayed in G1 phase like cord blood-derived vascular endothelial progenitor cells even though most of undifferentiated human embryonic stem cells stay in S phase. It was also identified that the expression of an undifferentiation marker SSEA-4 in the isolated vascular endothelial progenitor cells was 0.98% which is far lower than that in undifferentiated human embryonic stem cells (90.1%). In addition, it was identified that the isolated vascular endothelial progenitor cells were derived from CHA-3 human embryonic stem cells as a result of DNA fingerprinting.

1. A process for differentiation of vascular endothelial progenitor cells from embryoid bodies derived from embryonic stem cells, the process comprising:
(a) treating a culture medium comprising embryoid bodies derived from embryonic stem cells such that the concentration of oxygen dissolved in the culture medium is in the range of about 1 ppm to about 5 ppm;
(b) culturing the culture medium prepared in step (a) in an incubator in which the oxygen (O₂) tension is equal to or less than about 15% to differentiate the embryoid bodies into vascular endothelial progenitor cells; and
(c) isolating the vascular endothelial progenitor cells from the culture medium obtained in step (b).

2. The process of claim 1, wherein the concentration of oxygen dissolved in the culture medium is in the range of about 2 ppm to about 3 ppm in step (a).

3. The process of claim 1 or 2, wherein the treatment in step (a) is performed by bubbling an inert gas.

4. The process of claim 3, wherein the inert gas is argon (Ar), helium (He), or nitrogen (N₂) gas.

5. The process of claim 4, wherein the inert gas is nitrogen (N₂) gas.

6. The process of claim 1, wherein the oxygen tension is about 1% to about 10% in step (b).

7. The process of claim 1 or 6, wherein the culturing is performed for about 1 week to 3 weeks.

8. The process of claim 1, wherein the isolation in step (c) is performed using a fluorescence activated cell sorter (FACS).

9. The process of claim 8, wherein the isolation using the FACS is performed by isolating cells positive for both CD133 and KDR/Flk-1 as markers.
